(19) United States
(12) Patent Application Publication
(10) Pub. No.: US 2014/0094890 A1
(43) Pub. Date: Apr. 3, 2014

(54) IMPLANTABLE THERAPY LEAD WITH CONDUCTOR CONFIGURATION ENHANCING ABRASION RESISTANCE

(71) Applicant: PACESETTER, INC., Sylmar, CA (US)

(72) Inventors: Tyler Strang, Valencia, CA (US); Daniel Hale, North Hollywood, CA (US); Michael Childers, Montrose, CA (US); Xiaoyi Min, Camarillo, CA (US); Wenbo Hou, Valencia, CA (US); Phong D. Doan, Stevenson Ranch, CA (US)

(73) Assignee: PACESETTER, INC., Sylmar, CA (US)

(21) Appl. No.: 13/766,597

(22) Filed: Feb. 13, 2013

Related U.S. Application Data
(63) Continuation of application No. 13/631,540, filed on Sep. 28, 2012.

(51) Int. Cl. A61N 1/05

(52) U.S. Cl. CPC A61N 1/0587
USPC 607/119

(57) ABSTRACT

An implantable therapy lead employs electrical conductors configured to enhance the abrasion resistance of the lead. Specifically, conductors are configured to create a surface contact area with walls of a wall lumen of a tubular body that is greater than would otherwise be possible with traditional conductors that have a circular transverse cross-section. As a result, the abrasion pressure of the conductors against the lumen walls is decreased for the conductors disclosed herein as compared to that of traditional conductors.

### CROSS REFERENCE TO RELATED APPLICATIONS

This is a continuation of U.S. patent application Ser. No. 13/631,540, filed Sep. 28, 2012.

### FIELD OF THE INVENTION

The present invention relates to medical apparatus and methods. More specifically, the present invention relates to implantable therapy leads and methods of manufacturing such leads.

### BACKGROUND OF THE INVENTION

Lead failure issues have become visible in the cardiac rhythm device industry. Clinical observations report finding conductors external to the lead body. The root cause for this type of lead failure is due to the silicone lead body wearing down from the inside of a conductor lumen and eventually resulting in a breach long enough for a conductor to become exposed. The driving force for the wear is the conductors experiencing repetitive motion due to the contractions of the heart placing the conductors into tension, thereby forcing the conductors to apply pressure to the inside of the wall of the respective conductor lumens.

There is a need in the art for a lead offering improved abrasion resistance without an increased diameter and reduced flexibility. There is also a need in the art for a method of manufacturing such a lead.

### SUMMARY

An implantable therapy lead is disclosed herein. In one embodiment, the lead includes a polymer tubular body and a conductor. The polymer tubular body includes a proximal end, a distal end, a length between the proximal and distal ends, a wall including an outer circumferential surface, and a wall lumen extending through the wall between the proximal and distal ends. The wall lumen is defined in the wall by a lumen wall surface forming an inner circumferential surface of the wall lumen.

In one version of the embodiment of the lead, the conductor extends through the wall lumen and includes a cross-section transverse to the length of the polymer tubular body. The cross-section includes a first transverse cross-sectional dimension terminating in first and second endpoints, a second transverse cross-sectional dimension greater than the first transverse cross-sectional dimension and ending in third and fourth endpoints, and an arcuate outer surface extending in a continuous, non-deviating manner between the third and fourth endpoints and through the first endpoint.

In another version of the embodiment of the lead, the conductor extends through the wall lumen and includes a cross-section transverse to the length of the polymer tubular body. The cross-section includes a first transverse cross-sectional dimension terminating in first and second endpoints, a second transverse cross-sectional dimension greater than the first transverse cross-sectional dimension and ending in third and fourth endpoints, and a straight outer surface extending in a continuous, non-deviating manner through the first endpoint.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a side view of a CRT system.

FIG. 2 is a transverse cross section of the lead tubular body as taken along section line 2-2 in FIG. 1.

FIG. 3 is a longitudinal cross section of the lead tubular body as taken along section line 3-3 in FIG. 2A.

FIG. 4A is a transverse cross-section of the conductor configuration depicted as employed in the lead tubular body of FIG. 2.

FIG. 4B is an isometric view of the conductor configuration depicted in FIG. 4A.

FIG. 5 is a transverse cross-section of an alternative conductor configuration that may be employed in the lead tubular body of FIG. 2.

FIG. 6 is a transverse cross-section of an alternative conductor configuration that may be employed in the lead tubular body of FIG. 2.

FIG. 7 is a transverse cross-section of an alternative conductor configuration that may be employed in the lead tubular body of FIG. 2.

FIG. 8A is a transverse cross-section of an alternative conductor configuration that may be employed in the lead tubular body of FIG. 2.

FIG. 8B is an isometric view of the conductor configuration depicted in FIG. 8A.

FIG. 9 is a transverse cross-section of an alternative conductor configuration that may be employed in the lead tubular body of FIG. 2.

FIG. 10 is a transverse cross-section of an alternative conductor configuration that may be employed in the lead tubular body of FIG. 2.

### DETAILED DESCRIPTION

### a) Overview

An implantable therapy lead 10 (e.g., a CRT lead, etc.) and a method of manufacturing such a lead are disclosed herein. The lead 10 employs electrical conductors 110 configured to enhance the abrasion resistance of the lead. Specifically, the conductors 110 are configured to create a surface contact area 135 with the walls 120 of the wall lumen 90 of the tubular body 22 that is greater than would otherwise be possible with traditional conductors that have a circular transverse cross-section. As a result, the abrasion pressure of the conductors 110 against the lumen walls 120 is decreased for the conductors 110 disclosed herein as compared to that of traditional conductors.

### b) Device

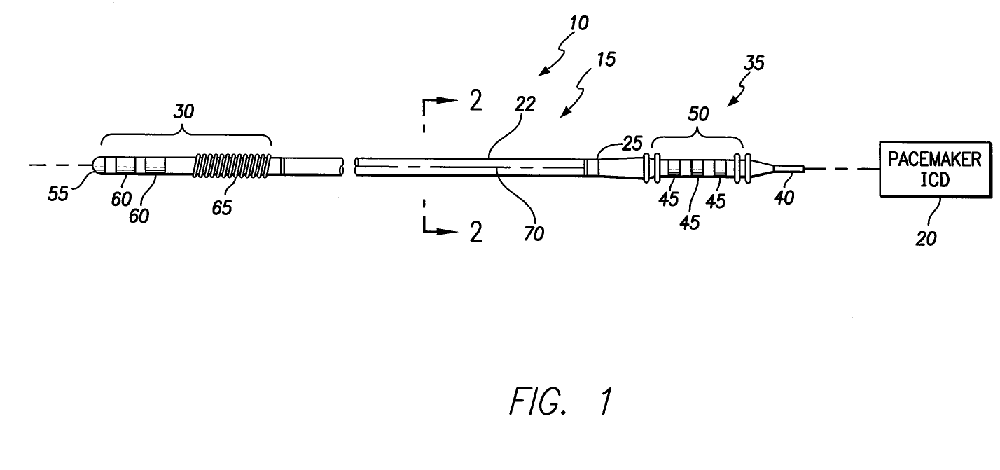

For a discussion regarding a CRT lead 10, reference is made to FIG. 1, which is a side view of a CRT system 10. As shown in FIG. 1, in one embodiment, the CRT system 10 includes a lead 15 and a pacemaker, defibrillator or ICD 20. In one embodiment, the lead 15 includes a tubular body 22 having a proximal end 25 and a distal end 30. In one embodiment, the lead 15 is of a quadripolar design, but in other embodiments the lead 15 will be of a design having a greater or lesser number of poles.

In one embodiment, the lead body 22 may be isodiametric, i.e., the outside diameter of the lead body 22 may be the same throughout its entire length. In one embodiment, the outside diameter of the lead body 22 may range from approximately 0.026 inch (2 French) to about 0.130 inch (10 French).

As depicted in FIG. 1, in one embodiment, a connector assembly 35 proximally extends from the proximal end 25 of the lead 15. In one embodiment, the connector assembly 35 is compatible with a standard such as the IS-4 standard for connecting the lead body to the ICD 20. The connector assembly 35 includes a tubular pin terminal contact 40 and ring terminal contacts 45. The connector assembly 22 of the lead 15 is received within a receptacle (not shown) in the ICD 20 containing electrical terminals positioned to engage the contacts 40, 45 on the connector assembly 35. As is well known in the art, to preventingress of body fluids into the receptacle, the connector assembly 35 is provided with spaced sets of seals 50. In accordance with standard implantation techniques, a stylet or guide wire (not shown) for delivering and steering the distal end of the lead body during implantation is inserted into a lumen of the lead body 22 through the tubular connector terminal pin 40.

As illustrated in FIG. 1, in one embodiment, the distal end 30 of the lead body 22 carries one or more electrodes 55, 60, 65 having configurations, functions and placements along the length of the distal end 30 dictated by the desired stimulation therapy, the peculiarities of the patient's anatomy, and so forth. The lead body 22 shown in FIG. 1 illustrates but one example of the various combinations of stimulating and/or sensing electrodes 55, 60, 65 that may be utilized.

As depicted in FIG. 1, in one embodiment, the distal end 30 of the lead body 22 includes one tip electrode 55, two ring electrodes 60 and a single cardioverting/defibrillating coil 65. The tip electrode 55 forms the distal termination of the lead body 22. The ring electrodes 60 are just distal of the tip electrode 55. The cardioverter/defibrillator coil 65 is just distal of the ring electrodes 60. Depending on the embodiment, the tip and ring electrodes 55, 60 may each serve as tissue-stimulating and/or sensing electrodes.

In other embodiments, other electrode arrangements will be employed. For example, in one embodiment, the electrode arrangement may include additional ring stimulation and/or sensing electrodes 60 as well as additional cardioverting and/or defibrillating coils 65 spaced apart along the distal end of the lead body 22. In one embodiment, the distal end 30 of the lead body 22 may carry only pacing and sensing electrodes, only cardioverting/defibrillating electrodes or a combination of pacing, sensing and cardioverting/defibrillating electrodes.

In conventional fashion, the distal end 30 of the lead body 22 may include passive fixation means (not shown) that may take the form of conventional projecting tines for anchoring the lead body within the right atrium or right ventricle of the heart. Alternatively, the passive fixation or anchoring means may comprise one or more preformed humps, spirals, S-shaped bends, or other configurations manufactured into the distal end 30 of the lead body 22 where the lead 15 is intended for left heart placement within a vessel of the coronary sinus region. The fixation means may also comprise an active fixation mechanism such as a helix. It will be evident to those skilled in the art that any combination of the foregoing fixation or anchoring means may be employed.

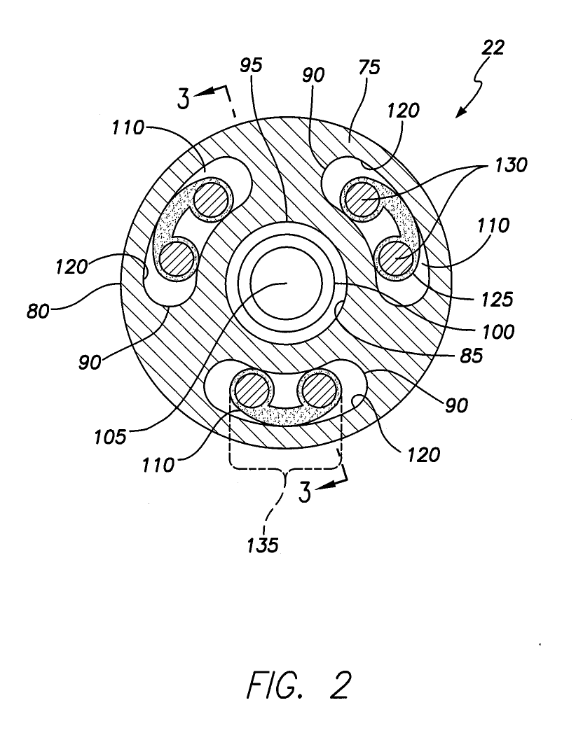

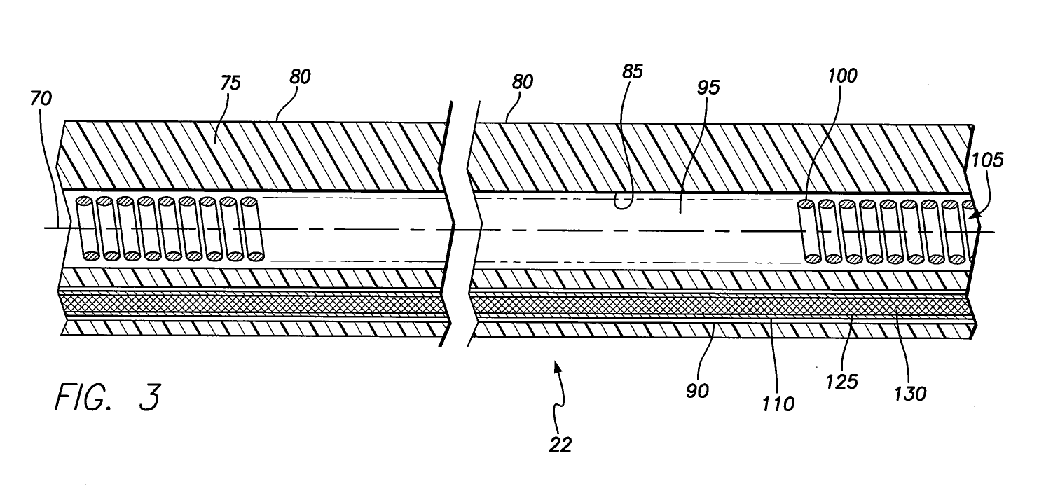

For a discussion regarding the construction of the tubular body 22 of the lead 15, reference is made to FIGS. 1, 2 and 3. FIG. 2 is a transverse cross section of the lead tubular body 22 as taken along section line 2-2 in FIG. 1. FIG. 3 is a longitudinal cross section of the lead tubular body 22 as taken along section line 3-3 in FIG. 2. As indicated in FIGS. 1 and 3, the lead body 22 extends along a central longitudinal axis 70.

As shown in FIGS. 2 and 3, the lead body 22 includes a wall 75 made of an insulating biocompatible biostable polymer (e.g., silicone rubber, polyurethane, SPC, etc.).

As depicted in FIGS. 2 and 3, the wall 75 includes an outer circumferential surface 80, an inner circumferential surface 85 and one or more wall lumens 90. In one embodiment, as illustrated in FIG. 2, the wall 75 has three arcuately or radially extending wall lumens 90. In other embodiments, the wall lumen will have other shapes (e.g., square, rectangular, circular, oval, etc.) and/or the wall 75 will have a greater or lesser number of wall lumens 90. Each wall lumen 90 is defined in the wall 75 via the walls 120 of the wall lumen 90.

In one embodiment, the wall lumens 90 extend generally linearly or straight through the length of the wall 75. In other embodiments, the wall lumens 90 extend generally helically or in a spiral through the length of the wall 75.

As indicated in FIGS. 2 and 3, in one embodiment, the outer circumferential surface 80 forms the overall outer circumferential surface of the lead body 22. In other embodiments, a jacket, layer, coating or sheath extends over the outer circumferential surface 80 to a greater or lesser extent. For example, in one embodiment and in accordance with well-known techniques, the outer surface of the lead body 22 may have a lubricious coating along its length to facilitate its movement through a lead delivery introducer and the patient's vascular system.

As shown in FIGS. 2 and 3, in one embodiment, the inner circumferential surface 85 defines a central lumen 95. In one embodiment, a helical coil 100 extends through the central lumen 95 and electrically connects the tubular connector terminal pin 40 with the tip electrode 55. The helical coil 100 defines a coil lumen 105 through which a stylet or guidewire can extend during implantation of the lead 15.

In one embodiment, the helical coil 100 is a helically coiled multi-filar braided cable formed of a metal such as stainless steel, Nitinol, platinum, platinum-iridium alloy, MP35N alloy, MP35N/Ag alloy, etc. In one embodiment, the helical coil is a helically coiled monofilament or single wire formed of a metal such as stainless steel, Nitinol, platinum, platinum-iridium alloy, MP35N alloy, MP35N/Ag alloy, etc.

In one embodiment, the central lumen 95 does not have a helical coil 100 extending through the central lumen 95. Instead, a liner made of a polymer such as PTFE extends through and lines the central lumen 95. Thus, the central lumen 95 has a slick or lubricious surface for facilitating the passage of the guidewire or stylet through the central lumen 95.

As shown in FIGS. 2 and 3, in one embodiment, each wall lumen 90 includes one or more electrical conductors 110 located within the confines of the wall lumen 90 defined by the wall 120 of the lumen 90. In one embodiment, each conductor 110 may have one or more electrically conductive cores 130. In some embodiments, a conductor 110 may have a polymer insulation layer or jacket 125 extending about the one or more electrically conductive cores 130 so as to electrically insulate the one or more cores 130 from the surroundings. In other embodiments, a conductor 110 may simply be the electrically conductive core 130 without a polymer insulation layer or jacket 125, the electrical isolation of the core 130 depending on the core 130 being electrically isolated from its surroundings via wall 120 of the lumen 90 containing the core 130.

In one embodiment, the one or more electrically conductive cores 130 of a conductor 110 is a multi-filar braided or helically wound cable formed of a metal such as stainless steel, platinum, platinum-iridium alloy, Nitinol, MP35N alloy, MP35N/Ag alloy, or etc. In one embodiment, the core 130 of a conductor 110 is a mono-filament non-coiled wire formed of a metal such as stainless steel, platinum, platinum-iridium alloy, Nitinol, MP35N alloy, MP35N/Ag alloy, or etc.

As can be understood from FIGS. 1, 2 and 3, in one embodiment, two of the conductors 110 respectively electrically connect two of the ring terminal contacts 45 to the two ring electrodes 60, and the third conductor 110 electrically connects the third ring terminal contact 45 to the cardioverter/defibrillator coil 65.

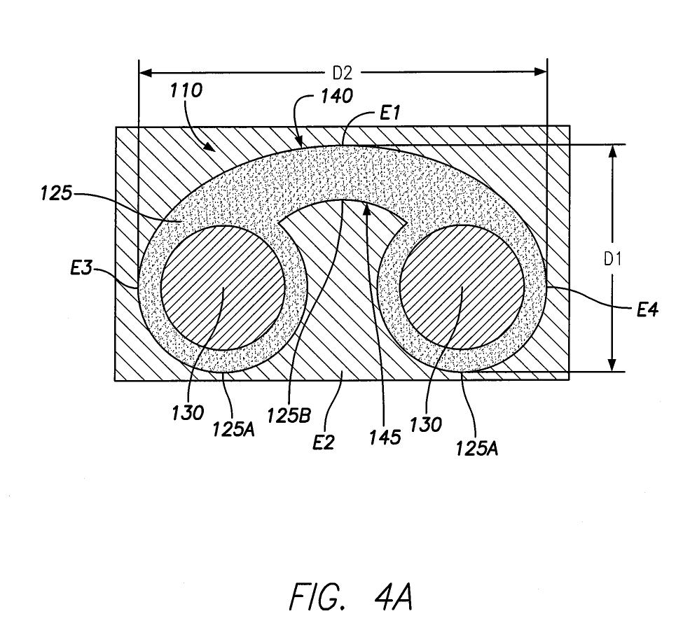

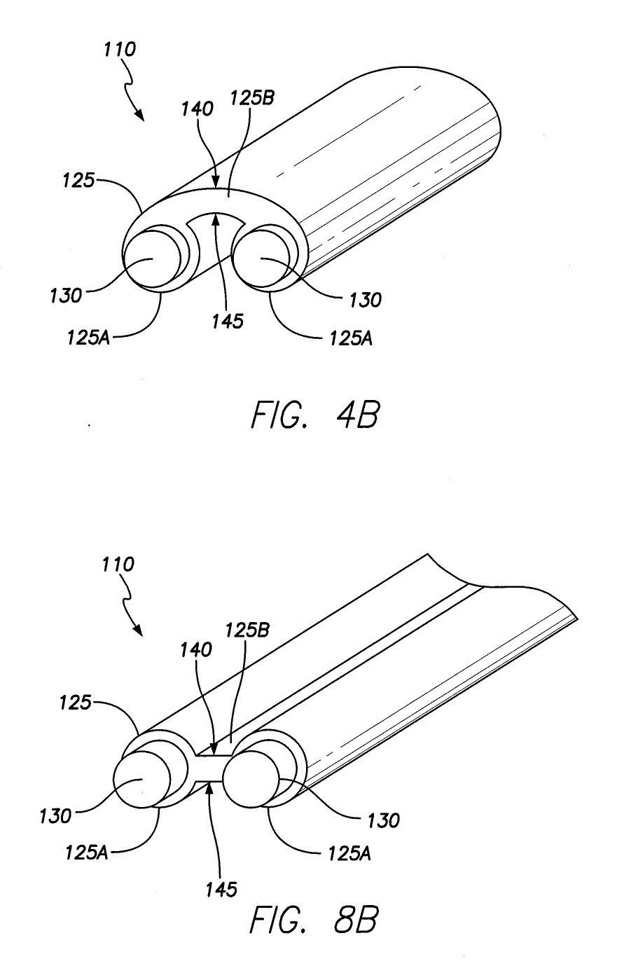

As can be understood from FIG. 2, in one embodiment, one or more, and even all, of the electrical conductors 110 extending through the lead tubular body 22 are configured to enhance the abrasion resistance of the lead. Specifically, a conductor 110 may be configured to create a surface contact area 135 with the wall 120 of the wall lumen 90 in which the conductor 110 resides that is greater than would otherwise be possible with a traditional conductor that has a circular transverse cross-section. For example, as indicated in FIGS. 4A and 4B, which are, respectively an enlarged transverse cross-sectional view and an enlarged isometric view of the conductor configuration depicted as employed in the wall lumens 90 of the tubular body 22 of FIG. 2, the conductor includes two electrically conductive cores 130 and an insulation layer or jacket 125. The cores 130 may have circular transverse cross-sections and are spaced apart from each other by a distance approximately equal to a diameter of one of the cores 130. The insulation layer 125 includes three portions, which are two circular portions 125A that each extend circumferentially about a respective outer circumference of a core 130 and a bridge portion 125B extending in an arcuate fashion between the two circular portions 125A.

Depending on the embodiment, to reduce abrasion between the conductors 110 and the tubular body wall 75, the insulation layer 125 may be formed of polytetrafluoroethylene (“PTFE”) or ethylene tetrafluoroethylene (“ETFE”). The outer surface of the insulation layer 125 may be coated with a hydrophilic coating. The insulation layer 125 may be employ nanoparticle technology such as, for example, being dry coated or impregnated with WS2 nanoparticles.

Depending on the embodiment, to reduce abrasion between the conductors 110 and the tubular body wall 75, the walls 120 of the wall lumens 90 may be formed of, or lined with, polytetrafluoroethylene (“PTFE”) or ethylene tetrafluoroethylene (“ETFE”). The exposed inner surface of the walls 120 of the wall lumens 90 may be coated with a hydrophilic coating. The exposed inner surface of the walls 120 of the wall lumens 90 may employ nanoparticle technology such as, for example, being dry coated or impregnated with WS2 nanoparticles.

Depending on the version of any of the conductor embodiments discussed below with respect to FIGS. 4A-10 and regardless of whether illustrated in a specific figure or not, each electrically conductive core 130 may have its own electrical insulation jacket 133 in addition to the insulation layer 125 extending about the core 130. Such insulation jackets 133 may be formed of PTFE, ETFE or other electrical insulation material. Conversely, depending on the version of any of the conductor embodiments discussed below with respect to FIGS. 4A-10 and regardless of whether illustrated in a specific figure or not, each electrically conductive core 130 may be free of any individual dedicated electrical insulation jacket 133 and simply rely on the electrical insulation provided by the insulation layer 125 or the surround wall lumen 90.

As can be understood from FIGS. 1, 2, 3 and 4A, a conductor 110 extends through the wall lumen 90 and includes a cross-section transverse to the length of the polymer tubular body 22. The transverse cross-section of the conductor 110 includes a first transverse cross-sectional dimension D1 terminating in first and second endpoints E1 and E2. The transverse cross-section of the conductor 110 also includes a second transverse cross-sectional dimension D2 greater than the first transverse cross-sectional dimension D1 and ending in third and fourth endpoints E3 and E4. In one embodiment, the first cross-sectional dimension D1 may be between approximately 0.152 mm and approximately 0.635 mm, and the second cross-sectional dimension D2 may be between approximately 0.305 mm and approximately 1.27 mm.

As illustrated in FIG. 4A, the bridge portion 125B extends between the two circular portions 125A and 125A such that an arcuate outer surface 140 of the insulation layer 125 and, more specifically, the bridge portion 125B, extends in a continuous, non-deviating arcuate manner between the third and fourth endpoints E3 and E4 and through the first endpoint E1.

As shown in FIG. 4A, the bridge portion 125B of the insulation layer 125 includes the arcuate outer surface 140 and an arcuate inner surface 145 opposite the arcuate outer surface 140. The arcuate inner surface 145 has a smaller radius of curvature than the arcuate outer surface 140. In one embodiment, the inner surface 145 may be a straight, non-arcuate surface. The bridge portion 125B intersects each circular portion 125A and 125A at approximately the same location, which in one embodiment, can be described as between a two o'clock and ten o'clock position on an outer circumference of the circular portion 125A.

As can be understood from FIGS. 2, 4A and 4B, the conductor 110 is configured to create a surface contact area 135 with the wall 120 of the wall lumen 90 in which the conductor 110 resides that is greater than would otherwise be possible with a traditional conductor that has a circular transverse cross-section. This increased surface contact area 135 is made possible at least in part because of the extended, arcuate surface of the bridge portion 125B, which extends in a continuous, non-deviating arcuate manner between the third and fourth endpoints E3 and E4 and through the first endpoint E1.

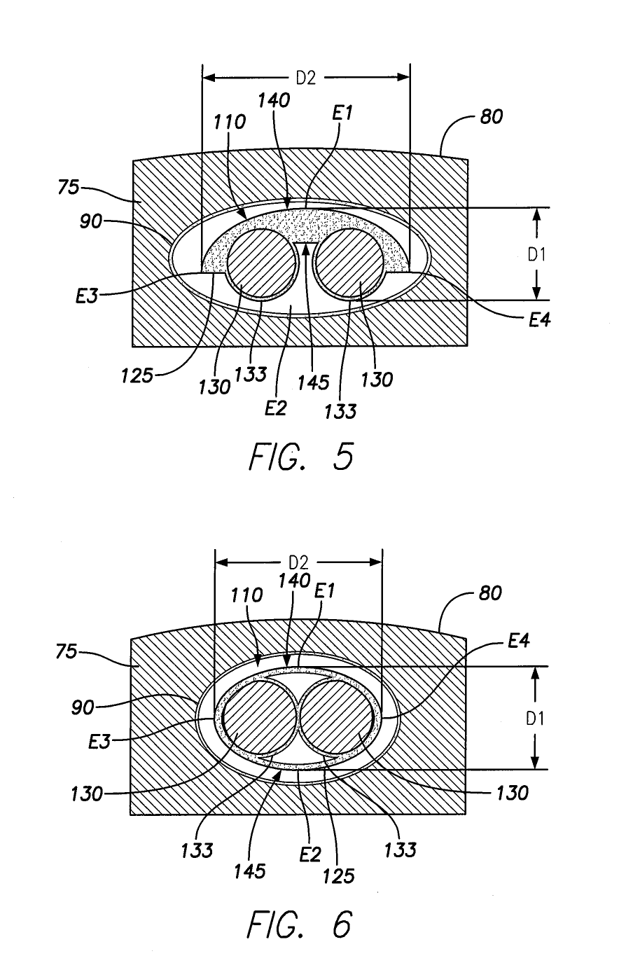

FIG. 5 is an enlarged transverse cross-section view of an another embodiment of a conductor 110 extending through a lumen 90 of the tubular body wall 75 near an outer circumferential surface 80 of the tubular body wall 75. Similar to the conductor embodiment discussed above with respect to FIGS. 4A and 4B, the conductor embodiment of FIG. 5 is configured to enhance the abrasion resistance of the lead by creating a surface contact area 135 (see FIG. 2) with the wall 120 of the wall lumen 90 in which the conductor 110 resides that is greater than would otherwise be possible with a traditional conductor that has a circular transverse cross-section.

As indicated in FIG. 5, the conductor 110 includes two electrically conductive cores 130 and an insulation layer or jacket 125. The cores 130 may have circular transverse cross-sections and are spaced apart from each other by a distance approximately equal to a quarter diameter of one of the cores 130. The insulation layer 125 includes a single portion, which may be considered a bridge portion extending in an arcuate fashion between the two cores 130. The insulation layer 125 does not have portions that extend circumferentially about the cores 130. Thus, the cores 130 are not insulated from each other or the surroundings via the insulation layer 125. Instead, the cores 130 may have their own individual insulation layers or jackets, or the cores 130 may be free of insulation within the confines of the lumen 90.

As can be understood from FIG. 5, the conductor 110 extends through the wall lumen 90 and includes a cross-section transverse to the length of the polymer tubular body 22. The transverse cross-section of the conductor 110 includes a first transverse cross-sectional dimension D1 terminating in first and second endpoints E1 and E2. The transverse cross-section of the conductor 110 also includes a second transverse cross-sectional dimension D2 greater than the first transverse cross-sectional dimension D1 and ending in third and fourth endpoints E3 and E4. In one embodiment, the first cross-sectional dimension D1 may be between approximately 0.152 mm and approximately 0.635 mm, and the second cross-sectional dimension D2 may be between approximately 0.305 mm and approximately 1.27 mm.

As illustrated in FIG. 5, the insulation layer 125 extends between the two cores 130 such that an arcuate outer surface 140 of the insulation layer 125 extends in a continuous, non-deviating arcuate manner between the third and fourth endpoints E3 and E4 and through the first endpoint E1.

As shown in FIG. 5, the insulation layer 125 includes the arcuate outer surface 140 and an inner surface 145 opposite the arcuate outer surface 140. The inner surface 145 may be straight as illustrated in FIG. 5 or, alternatively, may be arcuate similar to the conductor embodiment shown in FIG. 4A where the inner surface 145 has a smaller radius of curvature than the arcuate outer surface 140. The insulation layer 125 intersects each core 130 and 130 at approximately the same mirrored or opposite location, which in one embodiment, can be described as between a four-thirty o'clock and ten o'clock position on an outer circumference of the right core 130 and between an eight-thirty o'clock and two o'clock position on an outer circumference of the left core 130.

As can be understood from FIGS. 2 and 5, the conductor 110 is configured to create a surface contact area 135 with the wall 120 of the wall lumen 90 in which the conductor 110 resides that is greater than would otherwise be possible with a traditional conductor that has a circular transverse cross-section. This increased surface contact area 135 is made possible at least in part because of the extended, arcuate surface of the insulation layer 125, which extends in a continuous, non-deviating arcuate manner between the third and fourth endpoints E3 and E4 and through the first endpoint E1.

FIG. 6 is an enlarged transverse cross-section view of an another embodiment of a conductor 110 extending through a lumen 90 of the tubular body wall 75 near an outer circumferential surface 80 of the tubular body wall 75. Similar to the conductor embodiments discussed above with respect to FIGS. 4A, 4B and 5, the conductor embodiment of FIG. 6 is configured to enhance the abrasion resistance of the lead by creating a surface contact area 135 (see FIG. 2) with the wall 120 of the wall lumen 90 in which the conductor 110 resides that is greater than would otherwise be possible with a traditional conductor that has a circular transverse cross-section.

As indicated in FIG. 6, the conductor 110 includes two electrically conductive cores 130 and an insulation layer or jacket 125. The cores 130 may have circular transverse cross-sections and may abut against each other in a side-to-side manner. The insulation layer 125 includes a single portion extending in an arcuate fashion between the two cores 130. The insulation layer 125 extends circumferentially about the cores 130 so as to enclose the two cores 130 within the confines of the insulation layer 125. Thus, the cores 130 are not insulated from each other via the insulation layer 125, but are insulated from the surroundings via the insulation layer 125. The cores 130 may have their own individual insulation layers or jackets, or the cores 130 may be free of insulation within the confines of the insulation layer 125.

As can be understood from FIG. 6, the conductor 110 extends through the wall lumen 90 and includes a cross-section transverse to the length of the polymer tubular body 22. The transverse cross-section of the conductor 110 includes a first transverse cross-sectional dimension D1 terminating in first and second endpoints E1 and E2. The transverse cross-section of the conductor 110 also includes a second transverse cross-sectional dimension D2 greater than the first transverse cross-sectional dimension D1 and ending in third and fourth endpoints E3 and E4. In one embodiment, the first cross-sectional dimension D1 may be between approximately 0.152 mm and approximately 0.635 mm, and the second cross-sectional dimension D2 may be between approximately 0.305 mm and approximately 1.270 mm.

As illustrated in FIG. 6, the insulation layer 125 extends between the two cores 130 such that an arcuate surface 140 of the insulation layer 125 extends in a continuous, non-deviating arcuate manner between the third and fourth endpoints E3 and E4 and through the first endpoint E1, and another arcuate surface 145 of the insulation layer 125 extends in a continuous, non-deviating arcuate manner between the third and fourth endpoints E3 and E4 and through the second endpoint E2.

As shown in FIG. 6, the insulation layer 125 includes the arcuate outer surfaces 140 and 145 and may be in the form of a relatively thin-walled insulation jacket 125, the two conductors 130 and 130 being occupying the volume enclosed by the thin-walled insulation jacket. Where the insulation layer 125 is in the form of a thin-walled insulation jacket, the insulation layer 125 intersects each core 130 and 130 at approximately the same location, which in one embodiment, can be described as between a six o'clock and 12 o'clock position on an outer circumference of the core 130.

In one embodiment, the insulation layer 125 is not a thin-walled insulation jacket but is instead an insulation layer that occupies the entirety of the volume defined by the arcuate outer surfaces 140 and 145 depicted in FIG. 6 that is not occupied by the cores 130 and 130 themselves. Thus, the cores 130 and 130 are embedded in the insulation layer 125 such that the material of the insulation layer 125 generally contacts approximately 100 percent of the outer circumferential surface of each core 130.

As can be understood from FIGS. 2 and 6, the conductor 110 is configured to create a surface contact area 135 with the wall 120 of the wall lumen 90 in which the conductor 110 resides that is greater than would otherwise be possible with a traditional conductor that has a circular transverse cross-section. This increased surface contact area 135 is made possible at least in part because of the extended, arcuate surfaces 140 and 145 of the insulation layer 125, which extends in a continuous, non-deviating arcuate manner between the third and fourth endpoints E3 and E4 and through the first and second endpoints E1 and E2. Where the insulation layer 125 has an oval cross-section, the two arcuate surfaces 140 and 145 may smoothly and arcuately curve around the two cores 130 as a single generally continuous arcuate exterior surface.

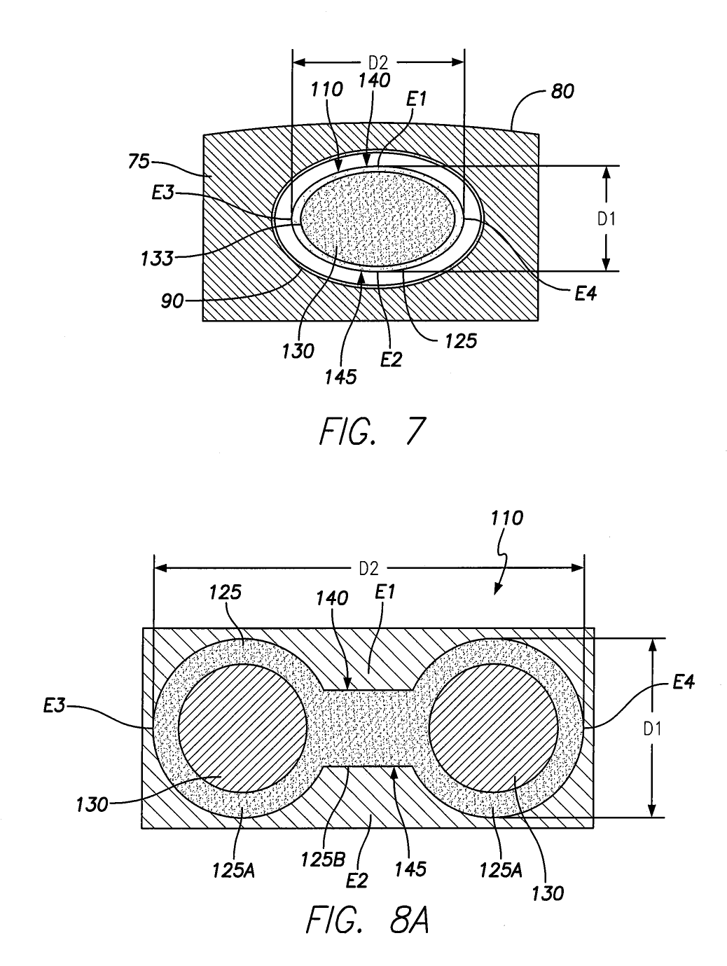

FIG. 7 is an enlarged transverse cross-section view of an another embodiment of a conductor 110 extending through a lumen 90 of the tubular body wall 75 near an outer circumferential surface 80 of the tubular body wall 75. Similar to the conductor embodiments discussed above with respect to FIGS. 4A, 4B, 5 and 6, the conductor embodiment of FIG. 7 is configured to enhance the abrasion resistance of the lead by creating a surface contact area 135 (see FIG. 2) with the wall 120 of the wall lumen 90 in which the conductor 110 resides that is greater than would otherwise be possible with a traditional conductor that has a circular transverse cross-section.

As indicated in FIG. 7, the conductor 110 includes a single electrically conductive core 130 and an insulation layer or jacket 125. The core 130 has a non-circular transverse cross-section such as, for example, an oval cross-section. The insulation layer 125 includes a single portion extending in an arcuate fashion about the core 130. The insulation layer 125 extends circumferentially about the core 130 so as to enclose the core 130 within the confines of the insulation layer 125.

As can be understood from FIG. 7, the conductor 110 extends through the wall lumen 90 and includes a cross-section transverse to the length of the polymer tubular body 22. The transverse cross-section of the conductor 110 includes a first transverse cross-sectional dimension D1 terminating in first and second endpoints E1 and E2. The transverse cross-section of the conductor 110 also includes a second transverse cross-sectional dimension D2 greater than the first transverse cross-sectional dimension D1 and ending in third and fourth endpoints E3 and E4. In one embodiment, the first cross-sectional dimension D1 may be between approximately 0.152 mm and approximately 0.635 mm, and the second cross-sectional dimension D2 may be between approximately 0.305 mm and approximately 1.27 mm.

As illustrated in FIG. 7, the insulation layer 125 extends about the oval core 130 such that an arcuate outer surface 140 of the insulation layer 125 extends in a continuous, non-deviating arcuate manner between the third and fourth endpoints E3 and E4 and through the first endpoints E1, and another arcuate surface 145 of the insulation layer 125 extends in a continuous, non-deviating arcuate manner between the third and fourth endpoints E3 and E4 and through the second endpoint E2. The core 130 is embedded or encased in the insulation layer 125 such that the material of the insulation layer 125 generally contacts approximately 100 percent of the outer circumferential surface of the core 130.

As can be understood from FIGS. 2 and 7, the conductor 110 is configured to create a surface contact area 135 with the wall 120 of the wall lumen 90 in which the conductor 110 resides that is greater than would otherwise be possible with a traditional conductor that has a circular transverse cross-section. This increased surface contact area 135 is made possible at least in part because of the extended, arcuate surfaces 140 and 145 of the insulation layer 125, which extends in a continuous, non-deviating arcuate manner between the third and fourth endpoints E3 and E4 and through the first and second endpoints E1 and E2. Where the insulation layer 125 has an oval cross-section, the two arcuate surfaces 140 and 145 may smoothly and arcuately curve around the single oval core 130 as a single generally continuous arcuate exterior surface.

For each of the conductor embodiments depicted in FIGS. 4A-7, it can be understood that the conductors 110 are oriented in the lumens 90 such that the arcuate outer surface 140 faces radially outward towards the outer circumferential surface 80 of the tubular lead body 22. Thus, the increased surface contact area 135 (see FIG. 2) exists where the conductors 110 are most likely to result in a failure in the tubular body wall 75, thereby reducing the likelihood of failure as compared to employing a conductor with a circular cross-section.

In one embodiment, the conductor 110 may employ two cores 130 joined together via a generally straight bridge portion 125B of the insulation layer 125. For example, as indicated in FIGS. 8A and 8B, which are, respectively an enlarged transverse cross-sectional view and an enlarged isometric view of the conductor configuration employing the straight bridge portion 125B, the conductor includes two electrically conductive cores 130 and an insulation layer or jacket 125. The cores 130 may have circular transverse cross-sections and are spaced apart from each other by a distance approximately equal to a diameter of one of the cores 130. The insulation layer 125 includes three portions, which are two circular portions 125A that each extend circumferentially about a respective outer circumference of a core 130 and a bridge portion 125B extending in straight, direct fashion between the two circular portions 125A.

As can be understood from FIGS. 1, 2, 3, 8A and 8B, a conductor 110 extends through the wall lumen 90 and includes a cross-section transverse to the length of the polymer tubular body 22. The transverse cross-section of the conductor 110 includes a first transverse cross-sectional dimension D1 terminating in first and second endpoints E1 and E2. The transverse cross-section of the conductor 110 also includes a second transverse cross-sectional dimension D2 greater than the first transverse cross-sectional dimension D1 and ending in third and fourth endpoints E3 and E4. In one embodiment, the first cross-sectional dimension D1 may be between approximately 0.152 mm and approximately 0.635 mm, and the second cross-sectional dimension D2 may be between approximately 0.305 mm and approximately 1.27 mm.

As illustrated in FIG. 8A, the bridge portion 125B extends between the two circular portions 125A and 125A in a continuous, non-deviating straight manner. The bridge portion 125B of the insulation layer 125 includes a straight outer surface 140 and a straight inner surface 145 opposite the straight outer surface 140. The bridge portion 125B intersects each circular portion 125A and 125A at approximately the same mirrored or opposite location, which in one embodiment, can be described as a three o'clock position on an outer circumference of the left circular portion 125A and a nine o'clock position on an outer circumference of the right circular portion 125A. The straight outer surface 140 has a length that is generally equal to the length of the straight inner surface 145.

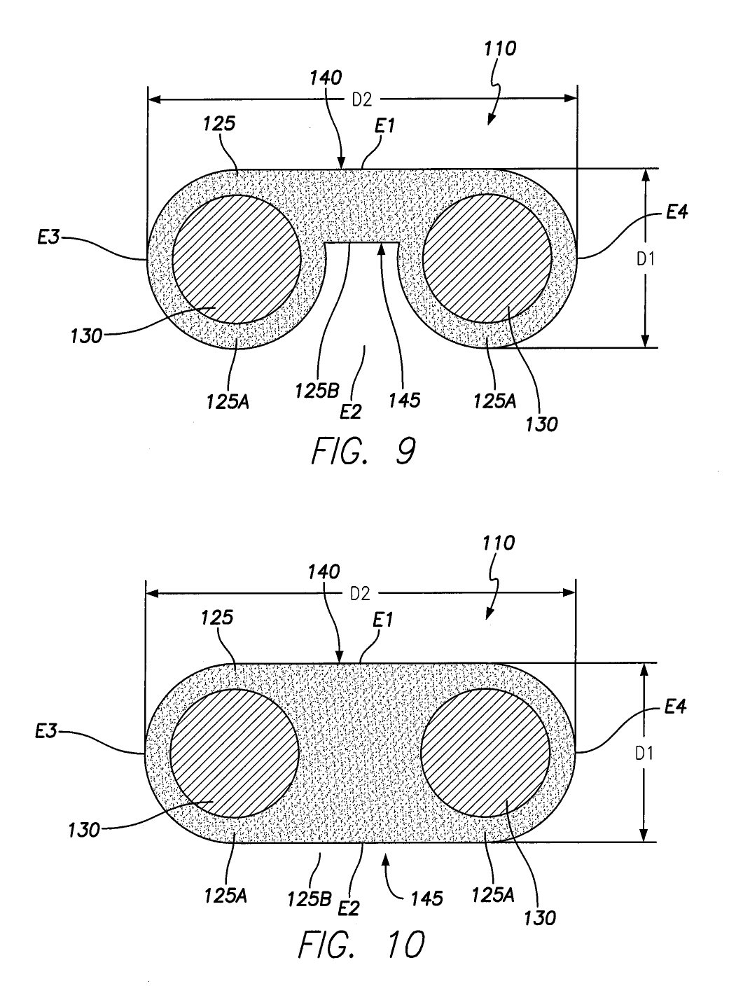

In an alternative embodiment, as depicted in FIG. 9, the bridge portion 125B extends between the two circular portions 125A and 125A in a continuous, non-deviating straight manner and is positioned such that the straight outer surface 140 is generally tangential with the outer circumferential surfaces of the two circular portions 125A and 125A, the straight inner surface 145 intersecting the outer circumferential surfaces of the two circular portions 125A and 125A in a non-tangential manner and, in some embodiments, in a generally normal or perpendicular manner. The bridge portion 125B intersects each circular portion 125A and 125A at approximately the same mirrored or opposite location, which in one embodiment, can be described as between a twelve o'clock position and a two-thirty o'clock position on an outer circumference of the left circular portion 125A and between twelve o'clock position and a nine-thirty o'clock position on an outer circumference of the right circular portion 125A. The straight outer surface 140 has a length that is greater than straight inner surface 145.

In yet another alternative embodiment, as depicted in FIG. 10, the bridge portion 125B extends between the two circular portions 125A and 125A in a continuous, non-deviating straight manner and is positioned such that the straight outer surface 140 is generally tangential with the outer circumferential surfaces of the two circular portions 125A and 125A, and the straight inner surface 145 is generally tangential with the outer circumferential surfaces of the two circular portions 125A and 125A. The bridge portion 125B intersects each circular portion 125A and 125A at approximately the same location, which in one embodiment, can be described as between a twelve o'clock position and a six o'clock position of the two circular portions 125A and 125A. In one embodiment, the bridge portion 125B and the two circular portions 125A and 125A may be a single unitary structure in which the two cores 130 and 130 are embedded.

As can be understood from FIGS. 2, 8A-10, the conductor 110 is configured to create a surface contact area 135 with the wall 120 of the wall lumen 90 in which the conductor 110 resides that is greater than would otherwise be possible with a traditional conductor that has a circular transverse cross-section. This increased surface contact area 135 is made possible at least in part because of the extended, straight surface of the bridge portion 125B, which extends in a continuous, non-deviating straight manner between the two circular portions 125A and 125A of the insulation layer 125.

### c) Method of Manufacture

A method of manufacturing the above-described lead 15 is now provided. As can be understood from FIGS. 2 and 3, in one embodiment, the wall 75 of the lead tubular body 22 is extruded or otherwise formed such that the wall lumens 90 are defined and established in the wall 75 and the wall inner circumferential surface 85 defines the central lumen 95. In one embodiment, the wall 75 is formed from a polymer material such as medical grade silicone rubber, polyurethane, or SPC. In one embodiment, the wall lumens 90 extend generally linearly or straight through the length of the wall 75. In other embodiments, the wall lumens 90 extend generally helically or in a spiral through the length of the wall 75.

As can be understood from FIGS. 2 and 3, in one embodiment, the helical coil 100 is placed into the central lumen 95, and the conductor cables 110 are placed into their respective wall lumens 90. In one embodiment, the helical coil 100 is fed into the central lumen 95. In other embodiments, the helical coil 100 is formed into the central lumen 95 or enters the central lumen 95 during extrusion of the wall 75. In one embodiment, the conductor cables 110 are fed into their respective wall lumens 90. In other embodiments, the conductor cables 110 are formed into their respective wall lumens 90 or enter their respective wall lumens 90 during extrusion of the wall 75.

In one embodiment, the lead body and its lumens are manufactured via a reflow process as known in the art.

Prior to being located within the wall lumens 90, the conductors having the various configurations described above with respect to FIGS. 4A-10 may be manufactured via various methods including, for example, extrusion of the insulation layer 125 about the core(s) 130.

Over the life of an implantable lead, the conductor cables 110 are sometimes in direct contact against the lumen walls 120, generating high stress in the wall insulation 75. Providing conductors 110 with configurations that provide increased surface contact area with the wall surfaces 120 of the lumens 120 containing the conductors 110 reduces the stress generated in the lumen wall surfaces 120 by the conductors contacting the wall surfaces 120. As a result, the frequency of tubular body failure or conductor failure on account of conductors breaking through the tubular body wall will decrease by employing the conductor configurations disclosed herein as compared to leads employing conductors having circular transverse cross-sections.

Although the present invention has been described with reference to preferred embodiments, persons skilled in the art will recognize that changes may be made in form and detail without departing from the spirit and scope of the invention.

What is claimed is:

1. An implantable therapy lead comprising:
a polymer tubular body comprising: a proximal end; a distal end; a length between the proximal and distal ends; a wall including an outer circumferential surface; and a wall lumen extending through the wall between the proximal and distal ends, the wall lumen defined in the wall by a lumen wall surface forming an inner circumferential surface of the wall lumen; and
a conductor extending through the wall lumen and comprising a cross-section transverse to the length of the polymer tubular body, the cross-section comprising: a first transverse cross-sectional dimension terminating in first and second endpoints; a second transverse cross-sectional dimension greater than the first transverse cross-sectional dimension and ending in third and fourth endpoints; and a straight outer surface extending in a continuous, non-deviating manner through the first endpoint.

2. The lead of claim 1, wherein the conductor further comprises a first electrically conductive core and a second electrically conductive core extending in a parallel manner through the conductor.

3. The lead of claim 2, wherein the first electrically conductive core and the second electrically conductive core extend in a parallel and spaced-apart manner through the conductor.

4. The lead of claim 2, wherein the first electrically conductive core and the second electrically conductive core extend in a parallel and abutting side-to-side manner through the conductor.

5. The lead of claim 2, wherein the conductor further comprises an insulation layer securing the first electrically conductive core to the second electrically conductive core and forming at least a portion of the straight outer surface that extends in a continuous, non-deviating manner through the first endpoint.

6. The lead of claim 5, wherein insulation layer further forms at least a portion of another straight outer surface extending in a continuous, non-deviating manner through the second endpoint.

7. The lead of claim 5, wherein the insulation layer includes:
a first circular portion that circumferentially extends about the first electrically conductive core;
a second circular portion that circumferentially extends about the second electrically conductive core; and
a bridge portion that extends between the first circular portion and the second circular portion and forms at least a portion of the straight outer surface that extends in a continuous, non-deviating manner through the first endpoint.

8. The lead of claim 7, wherein the bridge portion intersects the first circular portion and the second circular portion at generally the same location on each of the first and second circular portions.

9. The lead of claim 8, wherein the same location includes between approximately a twelve o'clock location and approximately a six o'clock position.

10. The lead of claim 7, wherein the bridge portion intersects the first circular portion and the second circular portion at generally the same mirrored or opposite location on each of the first and second circular portions.

11. The lead of claim 10, wherein the same mirrored or opposite location includes between approximately a two-thirty o'clock and approximately a twelve o'clock position on the first circular portion and between approximately a nine-thirty o'clock and approximately a twelve o'clock position on the second circular portion.

12. The lead of claim 1, wherein the wall further includes an inner circumferential surface, the polymer tubular body further comprises a central lumen defined by the inner circumferential surface, and the wall is located between the inner and outer circumferential surfaces.
